(19) United States
(12) Patent Application Publication
(10) Pub. No.: US 2023/0307136 A1
(43) Pub. Date: Sep. 28, 2023

(54) RISK ASSESSMENT SYSTEMS AND METHODS FOR PREDICTING AND REDUCING NEGATIVE HEALTH OUTCOMES ASSOCIATED WITH SOCIAL DETERMINANTS OF HEALTH

(71) Applicant: Experian Health, Inc., Franklin, TN (US)

(72) Inventors: Alan Tsang, San Diego, CA (US); Mustafa Adib, San Diego, CA (US); Shervin Sharifi, San Diego, CA (US); Mindy Pankoke, Lincoln, NE (US)

(21) Appl. No.: 17/702,611

(22) Filed: Mar. 23, 2022

(51) Int. Cl. G16H 50/30; G16H 50/70

(52) U.S. Cl. CPC G16H 50/30; G16H 50/70

(57) ABSTRACT

Embodiments of a risk assessment system is disclosed, where the risk assessment system can receive patient related data (for example, patient records) and social determinants of health data to generate a risk model to predict potential health outcomes, such as, for example readmission risk, with accompanying social determinants of health insight, which may include actionable insights and deployed systems that follow interventions. The risk model can be used to calculate a risk score for a patient based on patient records and social determinants of health data associated with the patient to predict potential negative health outcome for the patient proactively so that actions can be taken using social determinants of health insights to prevent that outcome.

### LIMITED COPYRIGHT AUTHORIZATION

A portion of the disclosure of this patent document includes material which is subject to copyright protection. The copyright owner has no objection to the facsimile reproduction by anyone of the patent document or the patent disclosure as it appears in the Patent and Trademark Office patent file or records, but otherwise reserves all copyrights whatsoever.

### FIELD OF EMBODIMENTS

This disclosure relates to a risk assessment platform environment for predicting negative health outcomes tied to social determinants of health.

### SUMMARY OF EMBODIMENTS

Various embodiments of systems, methods, and devices are disclosed for providing for predicting negative health outcomes, such as being readmitted to a care provider, and providing patient engagement strategies to prevent or reduce the risk of such negative health outcomes. The systems, methods, and devices of the disclosure each have several innovative aspects, no single one of which is solely responsible for the desirable attributes disclosed herein.

In one embodiment, a system for deployment of one or more risk models for assessing patient health outcome risk is provided. The system comprises: one or more processors; a network communications interface; a memory; and computer code stored in the memory, wherein the computer code, when retrieved from the memory and executed by the one or more processors causes the one or more processors to: access a first set of electronic patient data associated with a first patient, a provider admission, and an impending discharge event; automatically generate a linked patient data set based on the first set of electronic patient data and a unique identifier associated with the first patient where the unique identifier does not include any personally identifiable information of the first patient; associate the linked patient data set with one or more aggregated code indicators generated based on a set of anonymized clinical data comprising an aggregation of a plurality of anonymized provider data sets associated with a plurality of providers, wherein the set of anonymized clinical data includes risk rates for categorized clinical data; associate the linked patient data set with at least a length of stay based on an admission date associated with the provider admission; associate the linked data set with marketing attribute data comprising social determinants of health data; and apply a deployed risk model to generate a risk score associated with a health outcome risk based on the associated one or more aggregated code indicators or the associated marketing attribute data, where in the deployed risk model is generated based on the set of anonymized clinical data, where in the set of anonymized clinical data includes historical clinical data comprising historical data associated with the plurality of providers, historical provider admission data indicating dates of patient admissions associated with the plurality of providers, and historical discharge data indicating dates of patient discharge dates associated with the plurality of providers.

In a further embodiment, a computer-implemented method of deploying a risk model for assessing patient health outcome risk is provided. The computer-implemented method as implemented by one or more computing devices configured with specific executable instructions to at least: access a first set of electronic patient data associated with a first patient, a provider admission, and an impending discharge event; automatically generate a linked patient data set based on the first set of electronic patient data and a unique identifier associated with the first patient where the unique identifier does not include any personally identifiable information of the first patient; associate the linked patient data set with one or more aggregated code indicators generated based on a set of anonymized clinical data comprising an aggregation of a plurality of anonymized provider data sets associated with a plurality of providers, wherein the set of anonymized clinical data includes risk rates for categorized clinical data; associate the linked patient data set with at least a length of stay based on an admission date associated with the provider admission; associate the linked data set with marketing attribute data comprising social determinants of health data; and apply a deployed risk model to generate a risk score associated with a health outcome risk based on the associated one or more aggregated code indicators or the associated marketing attribute data, where in the deployed risk model is generated based on the set of anonymized clinical data, where in the set of anonymized clinical data includes historical clinical data comprising historical data associated with the plurality of providers, historical provider admission data indicating dates of patient admissions associated with the plurality of providers, and historical discharge data indicating dates of patient discharge dates associated with the plurality of providers.

In an additional embodiment, a non-transitory computer storage medium is provided. The non-statutory computer storage medium storing computer-executable instructions that, when executed by a processor, cause the processor to at least: access a first set of electronic patient data associated with a first patient, a provider admission, and an impending discharge event; automatically generate a linked patient data set based on the first set of electronic patient data and a unique identifier associated with the first patient where the unique identifier does not include any personally identifiable information of the first patient; associate the linked patient data set with one or more aggregated code indicators generated based on a set of anonymized clinical data comprising an aggregation of a plurality of anonymized provider data sets associated with a plurality of providers, wherein the set of anonymized clinical data includes risk rates for categorized clinical data; associate the linked patient data set with at least a length of stay based on an admission date associated with the provider admission; associate the linked data set with marketing attribute data comprising social determinants of health data; and apply a deployed risk model to generate a risk score associated with a health outcome risk based on the associated one or more aggregated code indicators or the associated marketing attribute data, where in the deployed risk model is generated based on the set of anonymized clinical data, where in the set of anonymized clinical data includes historical clinical data comprising historical data associated with the plurality of providers, historical provider admission data indicating dates of patient admissions associated with the plurality of providers, and historical discharge data indicating dates of patient discharge dates associated with the plurality of providers.

### BRIEF DESCRIPTION OF THE DRAWINGS

The foregoing aspects and many of the attendant advantages of this disclosure will become more readily appreciated as the same become better understood by reference to the following detailed description, when taken in conjunction with the accompanying drawings. The accompanying drawings, which are incorporated in, and constitute a part of, this specification, illustrate embodiments of the disclosure and may not be to scale.

Throughout the drawings, reference numbers are re-used to indicate correspondence between referenced elements. The drawings are provided to illustrate embodiments of the subject matter described herein and not to limit the scope thereof. Specific embodiments will be described with reference to the following drawings.

FIG. 1 is a system diagram showing an example embodiment of a readmission risk assessment environment using a readmission risk assessment system.

FIG. 2 is a block diagram illustrating an example embodiment of a readmission risk assessment system.

FIG. 3 is a workflow diagram schematically illustrating an example embodiment of a process of determining a patient readmission risk score.

FIGS. 4A and 4B are flow diagrams illustrating an example embodiment of a process for generating and deploying a readmission risk model.

FIG. 4C is a flow diagram illustrating an example embodiment of a process for calculating a patient admission risk score.

FIG. 5 is a flow diagram illustrating an example embodiment of a process for aggregating patient information.

FIG. 6 is a schematic diagram illustrating an example embodiment of a process for aggregating patient information.

FIG. 7 is a flow diagram illustrating an example embodiment of a process for assigning readmission tags.

FIG. 8 is a schematic diagram illustrating various scenarios for readmission tagging.

FIG. 9 is a flow diagram illustrating an example embodiment of a process for generating one or more risk tables.

FIG. 10 is a schematic diagram illustrating an example embodiment of a process for generating one or more risk tables.

FIG. 11 illustrates an example embodiment of a readmission risk table.

FIG. 12A is a flow diagram illustrating an example embodiment of a process for calculating a patient readmission risk score.

FIG. 12B is a flow diagram illustrating another example embodiment of a process for calculating a patient readmission risk score.

FIG. 13 is a flow diagram illustrating an example embodiment of a process for receiving patient information from an example service provider.

FIG. 14 illustrates an example user interface for displaying a patient readmission risk score and assessments for a number of factors for calculating patient readmission risk scores.

FIG. 15 a block diagram illustrating an example embodiment of a computing system.

### DETAILED DESCRIPTION OF EMBODIMENTS

Embodiments of the disclosure will now be described with reference to the accompanying figures. The terminology used in the description presented herein is not intended to be interpreted in any limited or restrictive manner, simply because it is being utilized in conjunction with a detailed description of embodiments of the disclosure. Furthermore, embodiments of the disclosure may include several novel features, no single one of which is solely responsible for its desirable attributes or which is essential to practicing the embodiments of the disclosure herein described. For purposes of this disclosure, certain aspects, advantages, and novel features of various embodiments are described herein. It is to be understood that not necessarily all such advantages may be achieved in accordance with any particular embodiment. Thus, for example, those skilled in the art will recognize that one embodiment may be carried out in a manner that achieves one advantage or group of advantages as taught herein without necessarily achieving other advantages as may be taught or suggested herein.

As mentioned above and as will now be explained in more detail and with reference to the drawings, this disclosure includes descriptions of embodiments of systems and methods for predicting negative health outcomes associated with a likelihood of readmission via a readmission risk platform. In some embodiments, the readmission risk platform may alert a provider that a patient treated by the provider has received a readmission risk score that indicates a strong likelihood that the patient is at risk for a potential negative health outcome and will be readmitted for further treatment by the provider. While some readmission risk may be associated solely with the patient's diagnosis, a large portion of readmission risk is associated with social determinants of health (SDOH) that cover the conditions and environments in which patients live. The SDOH may include, for example, nonclinical situations like lacking transportation, lacking financial means, lacking technological skills that hinder access to care, food, housing, or medication. Moreover, if caught early, negative health outcomes attributable to SDOH can often be prevented or reduced by including care that addresses those conditions and environments. As such, the readmission risk platform includes processes for linking and associating patients' data with SDOH aggregated criteria that allow the readmission risk platform to flag potential SDOH factors and provide alerts, actionable insights, and recommendations based on such SDOH aggregated criteria that are not available to provider systems. Moreover, in addition to or instead of readmission risk, the disclosure may be used for assessing other health outcomes, such as, for example, excessive acute or post-acute facility utilization, emergency room frequenter risk, high cost of care risk, and so forth, such that other health outcome risk models could be developed using the same methodology and systems. Various embodiments of systems and methods for providing a patient readmission risk assessment are disclosed in application Ser. No. 14/309,076 published as U.S. Publication No. 2014/0379363, and various embodiments of systems and methods for providing social determinants of health solutions are disclosed in application Ser. No. 16/916,747 published as U.S. Publication No. 2021/0035679, and both applications are hereby incorporated by reference herein in their entireties.

The readmission risk score may be calibrated for readmission within a predetermined timeframe, such as, for example, 30 days or 60 days, and may be based on a variety of factors including those attributable to SDOH using the SDOH aggregated criteria. In some embodiments, the readmission risk platform also provides actionable insights into why the patient has received a specific readmission risk score, such as for example, potential failure to adhere to a prescribed medication plan, high cost of care, excessive use of emergency services, likely no-show for follow-up patient visits, and so forth. In some embodiments, the readmission risk platform may also generate and provide recommendations for patient engagement strategies based on the readmission risk score or the actionable insights to help prevent the negative health outcome. In some embodiments, the readmission risk platform may be configured to provide one or more of the following to provide insight as to readmission risk, other health outcome, or other conditions risk: (a) within or in conjunction with care management, clinician user interfaces, or population health and similar care and healthcare analytics tools, (b) displayed as an alert when risk is over one or more risk thresholds, such as, a “high risk” threshold or “medium risk” threshold, and/or (c) as a widget that displays the score, action details, and recommendations, such as, in the form of a table, chart, or visualization of the solution in full or in part (score and/or insights into why and/or engagement strategy recommendations). It is recognized that information may be delivered via one or more of the following: (a) application programming interfaces (APIs), (b) Fast Healthcare Interoperability Resources (FHIR) portals, (c) Health Level 7 (HL7) portals, (d) text or email-based alerts to care team members, as HL7, and/or (e) other real-time push and pull feeds into provider, payer, and/or pharmacy portals.

### Patient Readmission Risk Assessment Environment

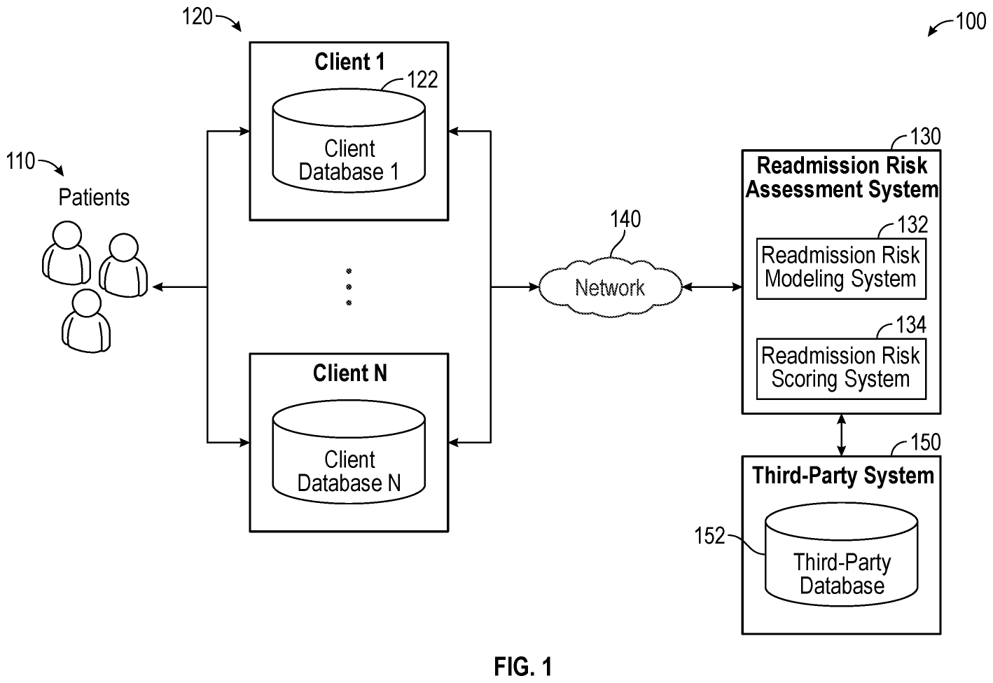

FIG. 1 is a system diagram illustrating an example embodiment of a patient readmission risk assessment environment 100 for calculating a likelihood of a given patient readmitted to a care provider. The patient readmission risk environment 100 shown in FIG. 1 can include patients 110, client systems 120, a readmission risk assessment system 130, a network, and a third-party system 150.

The patients 110 may be individuals who receive services (for example, patient care including inpatient and outpatient care) from the client systems 120. The client systems 120 may include systems utilized by care providers (for example, hospitals, doctors, surgeons, medical groups, urgent care centers, and other clinicians and providers) which admit the patients 110 and receive information related to the patients 110 (for example, diagnostics, length of admission, age, patient diagnostics history, gender, name, and so forth). The client systems 120 may include, for example, hospital IT platforms, health information systems (HIS), practice management systems (PMS), or the like. Based on the information related to the patients 110, data received from the client systems 120, and SDOH aggregated criteria, the patient readmission risk environment 100 may predict potential negative health outcomes for a given patient and determine a likelihood of the patient coming back to the provider within a certain amount of time (for example, 30 days, 60 days, 10 days, and so forth). Identifying potential patients who are susceptible or likely to be readmitted within a certain amount of time can be beneficial for the patients as such readmission is often associated with negative health outcomes as well as costs. Such negative health outcomes and costs, however, can be reduced (or prevented) by identifying patients who are likely readmitted within a certain amount of time (for example, 30 days) and providing preventive measures associated with SDOH factors (for example, more frequent care provider visitations or home monitoring) to reduce the likelihood of readmission. In some embodiments, the client systems 120 may determine the likelihood of readmission by themselves.

The client systems 120 can provide the patient information for a specific patient being discharged as well as collective patient information related to the patients 110 (for example, historical claims data) to the readmission risk assessment system 130 via the network 140. In addition to the patient information from the client systems 120, the readmission risk assessment system 130 can receive non-regulated information (for example, demographic information (such as age, geographic location, education level), financial information, credit information, housing information, and so forth) from a third-party database 152 operated by the third-party system 150.

Alternatively, while FIG. 1 shows the client systems 120 communicating with the readmission risk assessment system 130 via network 140, in some embodiments, the client systems 120 can include the readmission risk assessment system 130 to determine the likelihood of a given patient being readmitted within a certain amount of time within the client's firewalled client system 120.

In some embodiments, the patient information transmitted or made available by the client systems 120 to the readmission risk assessment system 130 may or may not be anonymized or scrubbed to remove any personally identifiable or sensitive information (for example, name, age, address, medical history information, and so forth). In some embodiments, the readmission risk assessment system 130 may undertake additional processes to ensure that the patient information received from the client systems 120 does not include any personally identifiable or sensitive information, or to abide local or federal requirements.

### Patient Readmission Risk Assessment System

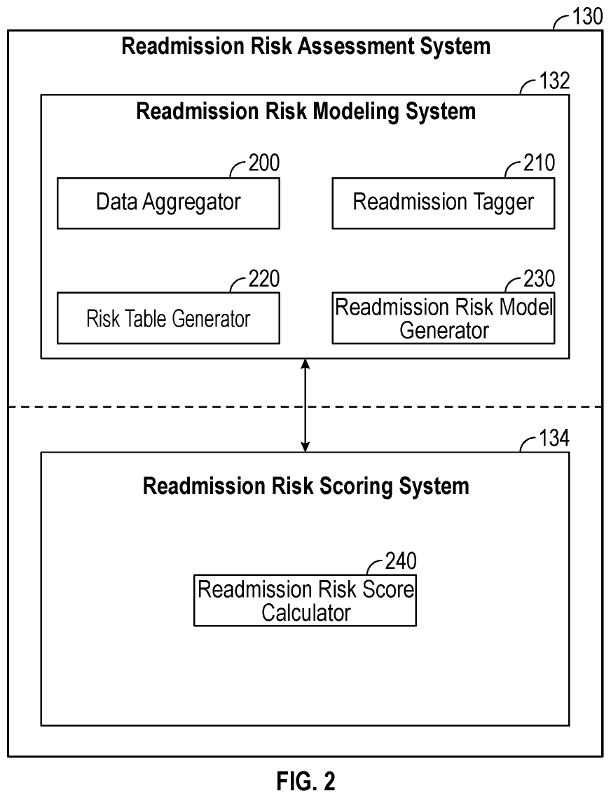

FIG. 2 is a block diagram illustrating an example embodiment of the patient readmission risk assessment system 130 for calculating a likelihood of a given patient readmitted to a care provider incorporating SDOH criteria. The patient readmission risk assessment system 130 can include a readmission risk modeling system 132 and a readmission risk scoring system 134.

Readmission Risk Assessment Modeling System

In some embodiments, the readmission risk modeling system 132 generates a readmission risk model based on historical patient data (including, for example, historical claims data) such that the readmission risk model predicts negative health outcomes for patients associated with readmission to a care provider incorporating SDOH criteria, and provides patient engagement strategies to prevent or reduce the risk of such negative health outcomes. In some embodiments, the readmission risk modeling system 132 generates a readmission risk model based on historical patient data of a specific provider, of a group of associated providers, or of a set of providers. The readmission risk modeling system 132 can include a data aggregator 200, a readmission tagger 210, a risk table generator 220, and a readmission risk model generator 230.

The data aggregator 200 can receive patient information from the client systems 120 for large sets of patients. The patient information may include for example, admission data, discharge data, diagnosis data, age, date of birth, residence address, county of residence, and so forth. For each of the patients in the patient information, the data aggregator 200 may associate each of the patient's data with a global identifier that can then be used to identify the patient information associated with each individual (for example, each patient of the patients 110) across multiple providers. This can be useful when an individual has visited a number of different care providers (for example, different clients of the client systems 120) and the readmission risk modeling system 132 receives information for the individual patient from multiple, different care providers. By identifying and aggregating information associated with the individual, the aggregator 200 allows the modeling system 130 to better understand patient admissions. In some embodiments, the data aggregator 200 may remove any personally identifiable information from the aggregated patient data such that the aggregated patient data links data associated with a specific patient across multiple providers, but does not include any information that identifies the specific patients.

After the data aggregator 200 aggregates the patient information and assigns identifiers for each patient, the readmission tagger 210 can tag instances of readmission for a given individual. For example, patient information for Patient A may include a list of admissions to one or more care providers (for example, hospital) and admission information associated with each of the admissions. In some embodiments, the admission information associated with the admission may include an admission date, reasons for admission, diagnosis, and a discharge date. In some embodiments, the readmission tagger 210 can identify instances of readmission across multiple providers based on the admission information.

The instances of readmission can be used to generate one or more risk tables and a readmission risk model. For example, the risk table generator 220 can use the instances of readmission (or readmission rate) and corresponding diagnostics information (of the instances of readmission) to determine correlations between patient diagnostics and the risk of readmission. In another example, marketing information (that is, received from the third-party system 150) can be used in conjunction with the patient information (for example, patient diagnostics) to determine correlations between patient diagnostics and/or marketing information and the risk of readmission. The readmission risk model generator 230 can use different correlations between the patient information (for example, diagnostics information) or the marketing information (for example, home address, income level, credit score, and so forth) and readmission rates to generate a readmission risk model for calculating a readmission risk score for a given individual (for example, a patient) based on patient information and/or marketing information associated with the given individual. In some embodiments, the readmission risk model generator 230 may include artificial intelligence such as neural networks, genetic algorithms, clustering, or the like. Machine learning may be performed using the aggregated patient data as a training set of data. The training data may be used to generate the readmission risk model that best characterizes a class of features of interest using the training data. In some embodiments, the class of features may be identified before training. In such embodiments, the readmission risk model may be trained to provide outputs most closely resembling the target class of features. In some implementations, no prior knowledge may be used or available for training the data. In such instances, the readmission risk model may discover new relationships for the provided training data. Such relationships may include similarities between data elements.

In some embodiments, the readmission risk modeling system 132 packages the risk tables and a readmission risk model for deployment.

Readmission Risk Assessment Scoring System

In some embodiments, the readmission risk assessment scoring system 134 generates a readmission risk model deploys the risk table and the readmission risk model for use by clients in determining a readmission risk score for a specific patient, such as, for example, at the time of the patient's discharge. The readmission risk assessment scoring system 134 may include a readmission risk score calculator 240 that receives information about the specific patient and generates an output package that includes the readmission risk score for the specific patient indicating a potential negative health outcome. The output package may also include actionable insights associated with SDOH that indicate why the patient has received the readmission risk score. In some embodiments, the output package may also include recommendations for patient engagement strategies based on the readmission risk score or the actionable insights to help prevent the negative health outcome. The readmission risk scoring system 134 may be implemented as part of the readmission risk modeling system 132, as a separate system of the readmission risk assessment system 130, as a stand-alone system separate from the readmission risk assessment system 130, or as part of one or more of the client systems 120.

### Process of Determining a Patient Readmission Risk Score

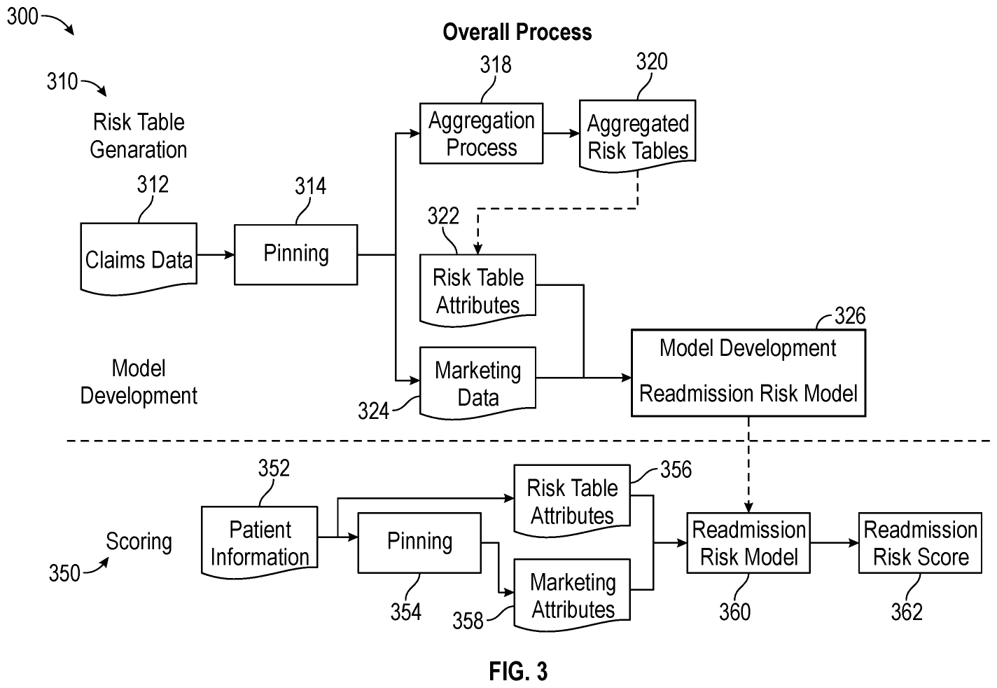

FIG. 3 is a schematic diagram illustrating an embodiment of a workflow 300 for determining a patient readmission risk score. The workflow 300 can include a model generation portion 310 to develop a readmission risk model for deployment and a scoring portion 350 that implements the readmission risk model as deployed.

The model generation portion 310 of the workflow 300 can begin with the readmission risk assessment system 130 receiving claims data at block 312. As discussed herein, the claims data can be historical claims data. However, it is recognized that the claims data may include other data related to the patient that may not be included in a specific claims data record, such as patient record data, diagnosis code data, payer data, and so forth. The readmission risk assessment system 130 may receive or access the claims data from the client systems 120 via the network 140. The claims data may be associated with patients 110 who receive service from the clients associated with the client systems 120. However, it is also recognized that the readmission risk model may be developed using claims data and/or patient data associated with one or more providers that are not associated with the client systems 120. After receiving or accessing the claims data, the readmission risk assessment system 130 can, at block 314, pin or link the claims data by, for example, associating claims data with unique non-personal identifiers associated with patients 110. At block 318, the readmission risk assessment system 130 may aggregate the linked or pinned claims data and generate one or more risk tables at block 320. At block 322, risk table attributes may be identified using the one or more risk tables generated at block 320. At block 324, the readmission risk assessment system 130 may receive non-regulated SDOH and other non-regulated data, such as demographic data or marketing data, from the third-party system 150. At block 326, the readmission risk assessment system 130 may develop a readmission risk model based on the risk table attributes and the non-regulated SDOH data.

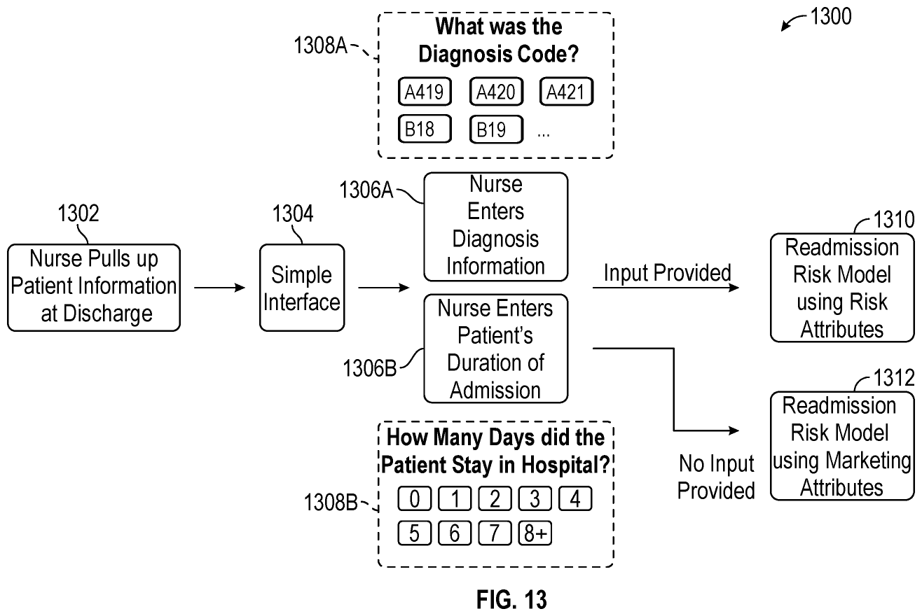

The scoring portion 350 of the workflow 300 can begin with the readmission risk assessment system 130 receiving patient information from the one of the client systems at block 352. For example, the patient information (for example, admission length and diagnosis) may be provided at discharge (see for example FIG. 13). The patient information may be linked or pinned at block 354 and linked with different non-regulated attributes associated with SDOH at block 358. For example, the patient information may be associated with a unique non-personal identifier, which may in turn be associated with SDOH data. By associating the patient information to the unique non-personal identifier, the patient information can be associated with the SDOH data. The patient information may be used identify risk table attributes (for example, by using the one or more risk tables generated at block 320). At block 360, the readmission risk model generated at block 326 may be used with the risk table attributes and the non-regulated attributes (for example, SDOH attributes or marketing attributes) to generate a readmission risk score at block 362. At block 360, the readmission risk model may also generate actionable insights associated with SDOH that indicate why the patient has received the readmission risk score as well as recommendations for patient engagement strategies based on the readmission risk score or the actionable insights to help prevent the negative health outcome for the patient.

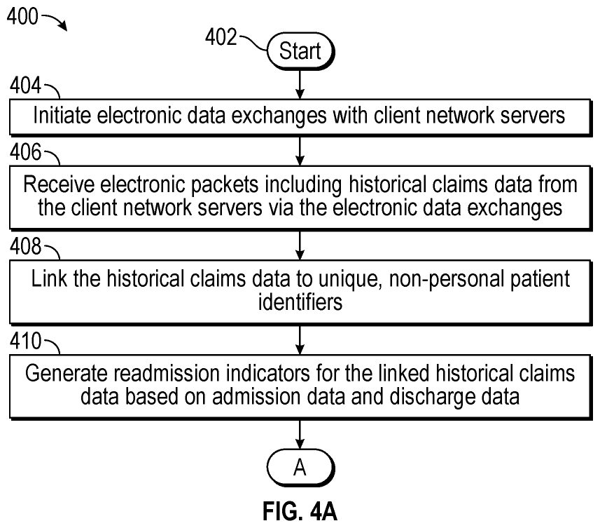

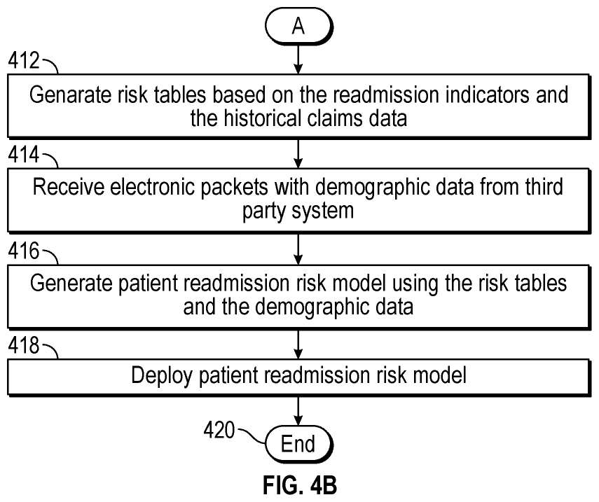

FIG. 4A and FIG. 4B are flow diagrams illustrating an example embodiment of a method 400 for generating and deploying a readmission risk model.

### Generating A Readmission Risk Model

The method 400 can begin at block 402. At block 404, the readmission risk assessment system 130 can initiate electronic data exchanges with client network servers operated by the client systems 120. At block 406, the readmission risk assessment system 130 can receive electronic packets that include historical claims data (for example, as a part of patient information) from the client network servers via the electronic data exchanges. The historical claims data can include admission information (for example, an admission date, a discharge date, county of residence, zip code of residence, age, and so forth), diagnostic information, and so forth. An example of the historical claims data from different clients is shown Tables 1 and 2 below. While example embodiments are being shown in Tables 1 and 2 below, as well as in the other tables herein, it is recognized that such tables are only example embodiments that that a variety of embodiments may be used and that other and or different data may be included.

TABLE 1
Example Historical Claims Data for a First Provider
Claims Data - Hospital A

| | | | | | Primary | | | |
|---|---|---|---|---|---|---|---|---|
| Client | First | Last | | | Diagnosis | | Discharge | |
| ID | Name | Name | Date of Birth | County | Code | Admit Date | Date | . . . |
| H-A | John | Smith | May 11, 1980 | LA | C2039 Sepsis | Sep. 1, 2019 | Sep. 6, 2019 | . . . |
| H-A | Eli | Perez | Dec. 12, 2000 | LA | C2044 Liver | Jan. 10, 2018 | Jan. 31, 2018 | |
| | | | | | Infection | | | |
| H-A | . . . | . . . | . . . | . . . | . . . | . . . | . . . | . . . |

TABLE 2
Example Historical Claims Data for a Second Provider
Claims Data - Hospital B

| | | | | | Primary | | | |
|---|---|---|---|---|---|---|---|---|
| Client | First | Last | | | Diagnosis | | Discharge | |
| ID | Name | Name | Date of Birth | County | Code | Admit Date | Date | . . . |
| H-B | J. | Smith | May 11, 1980 | LA | D2839 | Sep. 20, 2019 | Sep. 21, 2019 | . . . |
| | | | | | Infection | | | |
| H-B | Nellie | Liu | Nov. 5, 1975 | LA | C2044 Liver | Jan. 10, 2018 | Jan. 31, 2018 | |
| | | | | | Infection | | | |
| H-B | . . . | . . . | . . . | . . . | . . . | . . . | . . . | . . . |

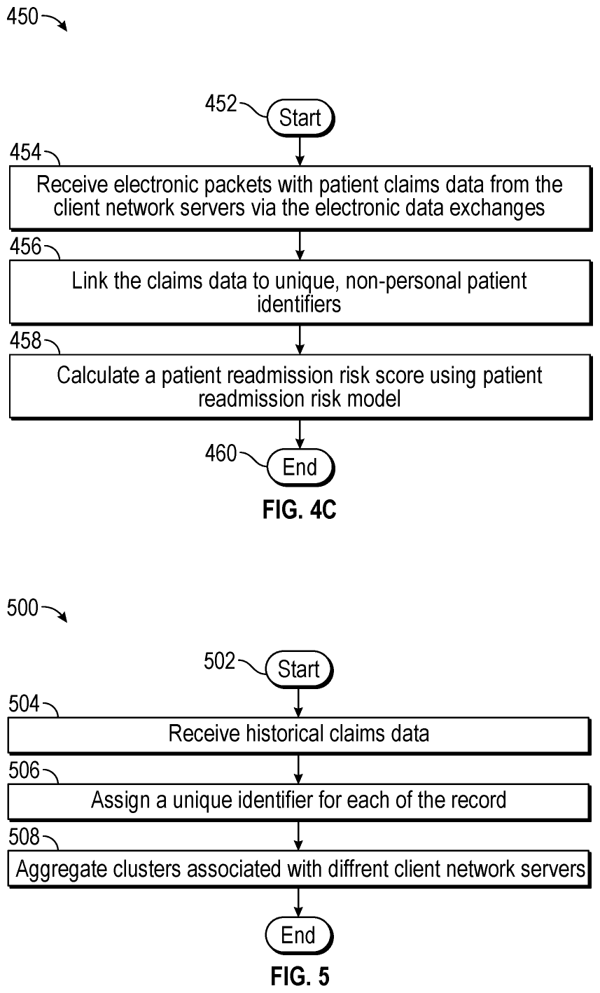

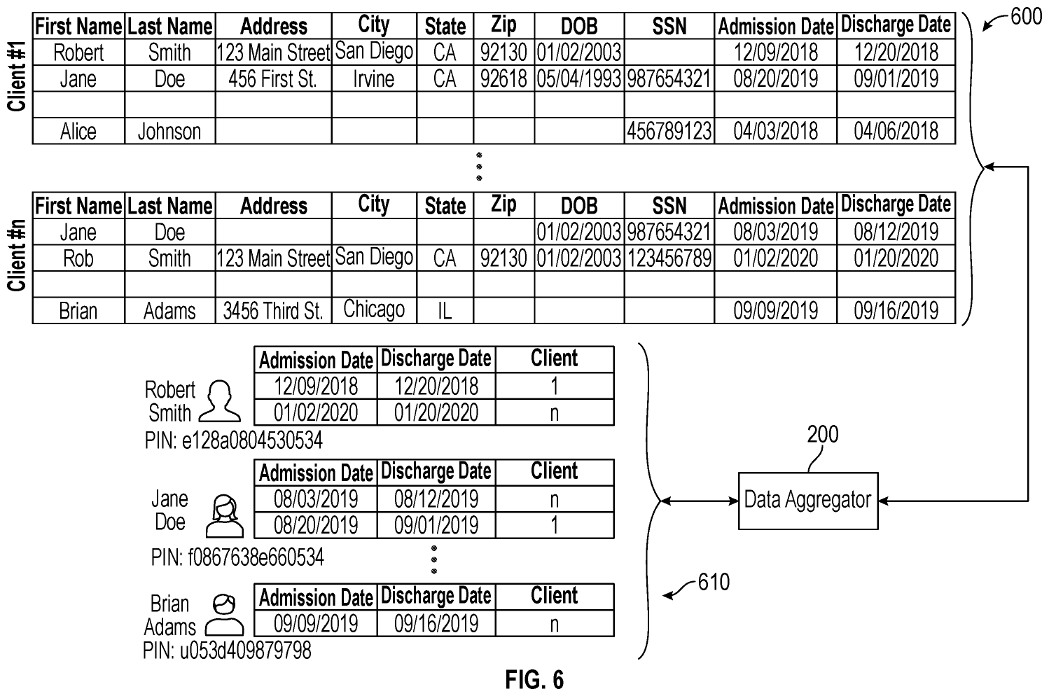

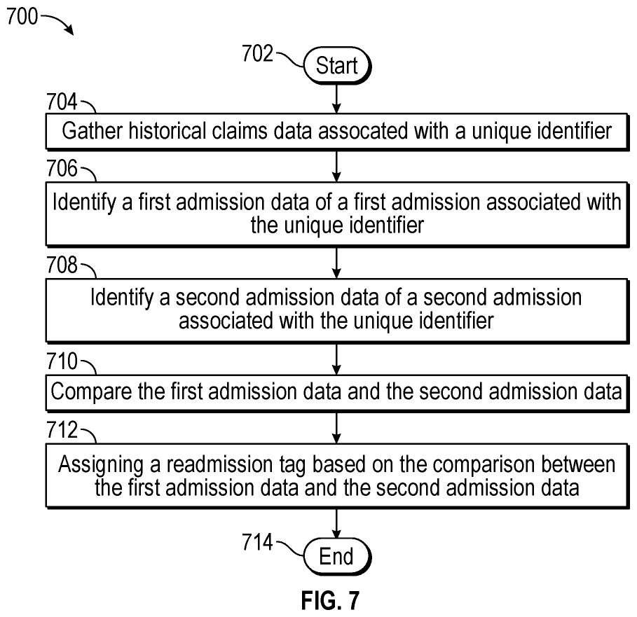

At block 408, the readmission risk assessment system 130 can link historical claims data to unique, non-personal patient identifiers. In some embodiments, the linking process can be performed using the embodiments of FIG. 5 and FIG. 6. After the readmission risk assessment system 130 receives or accesses historical claims data from the client systems 120, the claims data may not be aggregated across providers and linked. This linking across multiple providers may be advantageous because some patients may go to different care providers for receiving, for example, medical services. At block 410, the readmission risk assessment system 130 can generate readmission indicators for the linked historical claims data based on admission data (for example, admission date) and discharge data (for example, discharge date). In some embodiments, the generating of readmission indicators process can be performed using the embodiments of FIG. 7.

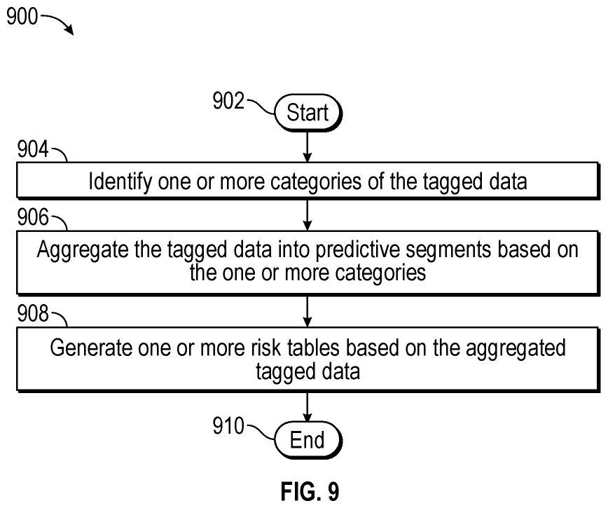

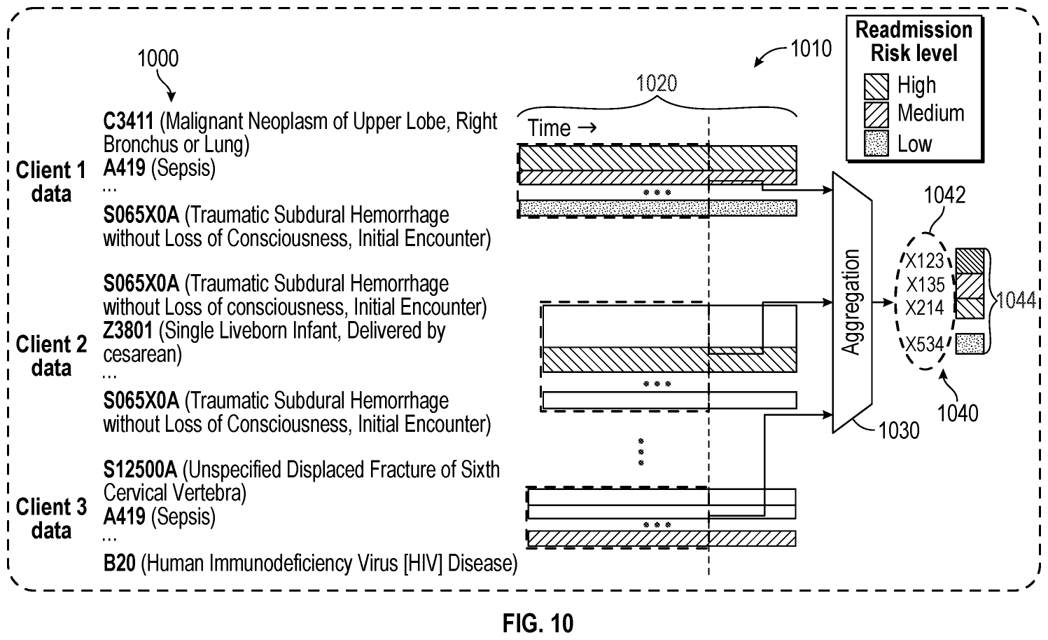

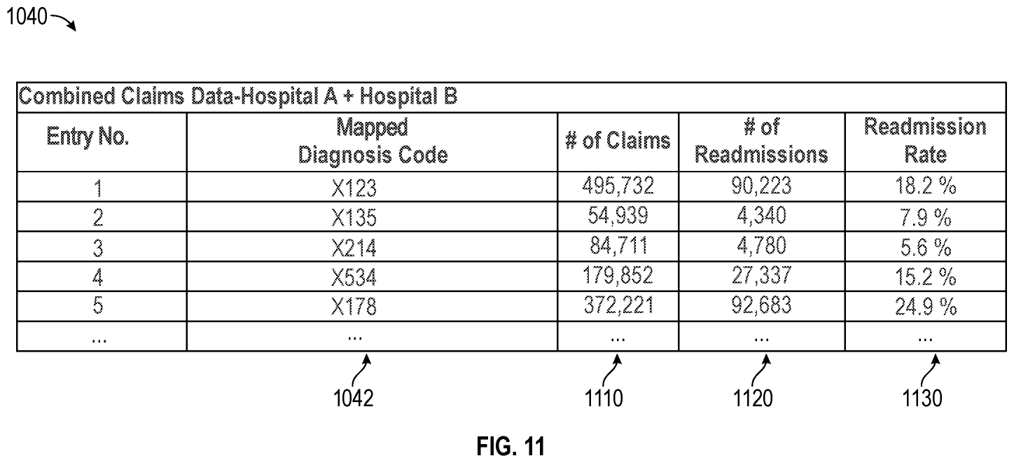

At block 412, the readmission risk assessment system 130 can generate one or more risk tables based on the readmission indicators and the historical claims data. For example, the risk tables can represent correlations between readmission rates and patient diagnostics information from the historical claims data. In some embodiments, the generating of risk tables process can be performed using the embodiments of FIG. 9, FIG. 10, and FIG. 11. At block 414, the readmission risk assessment system 130 can receive electronic packets including non-regulated SDOH data (for example, age, income level, credit score, amount of debt, home address, and so forth) from the third-party system 150 that may not be part of the historical claims data or other patient data accessible by the client systems 120. At block 416, the readmission risk assessment system 130 can generate patient readmission risk model using the one or more risk tables and the non-regulated SDOH data. The readmission risk model can indicate not only correlations between readmission risks and diagnostics information, but by incorporating the non-regulated SDOH data, the patient readmission risk model can take into account correlations between readmission risks and the conditions and environments in which patients live, such as, for example, nonclinical situations like lacking transportation, lacking financial means, lacking technological skills that hinder access to care, food, housing, or medication, and so forth). At block 418, the readmission risk assessment system 130 can deploy the readmission risk model. For example, the readmission risk assessment system 130 can deploy the readmission risk model on the readmission risk assessment system 350 or the readmission risk scoring system 134 such that the clients 120 can access the risk model via the network 140. In other embodiments, the readmission risk assessment system 130 can send or make available the readmission risk model to the client systems 120 via the network 140 such that the client systems 120 can run the readmission risk model on one of their processing devices. At block 420, the method 400 can end.

Deploying a Readmission Risk Model

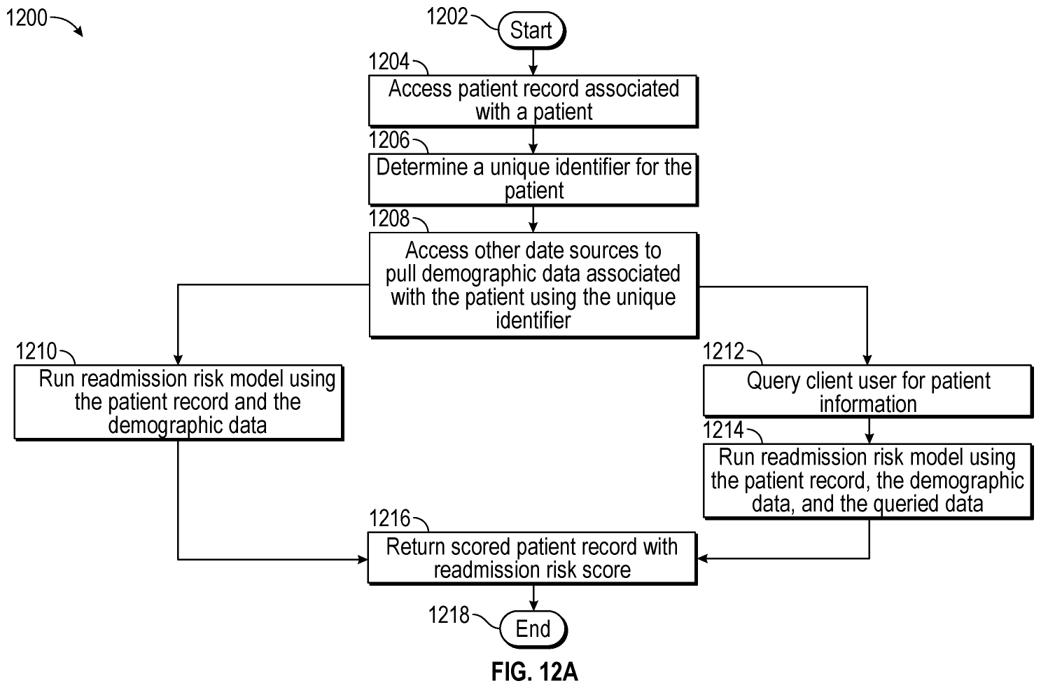

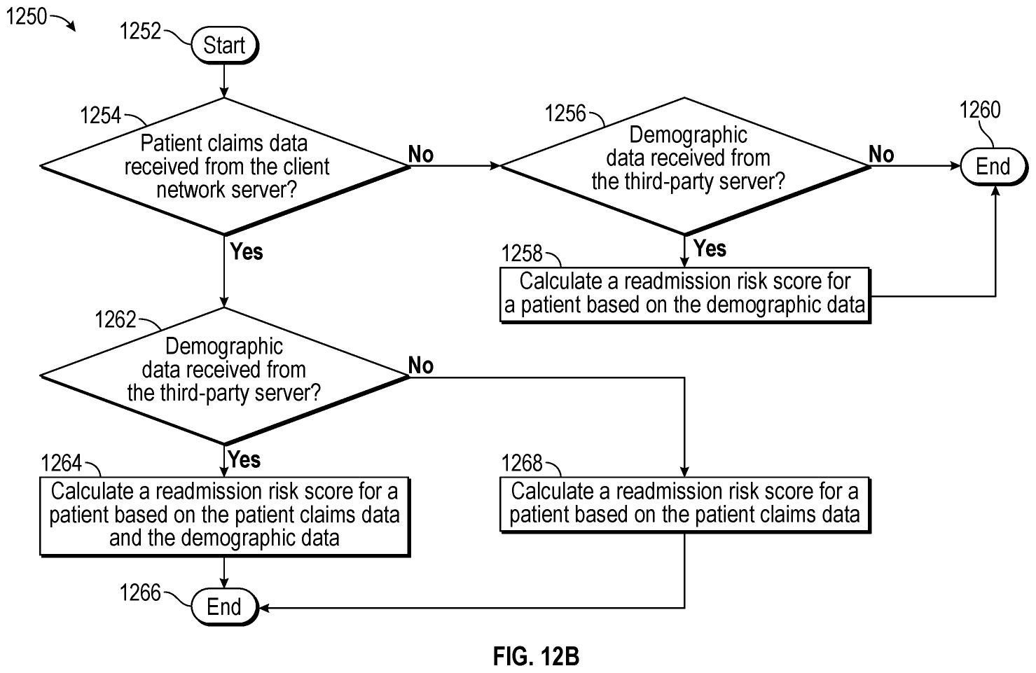

FIG. 4C is a flow diagram illustrating an example embodiment of a method 450 for calculating a patient admission risk score based on a deployed readmission risk model. The method 450 can begin at block 452. At block 454, the readmission risk assessment system 130 can receive an electronic packet including patient data (for example, patient data associated with a patient's recent admission) from client network servers (for example, servers associated with the client systems 120) via electronic data exchanges established between the client systems 120 and the readmission risk assessment system 130. At block 456, the readmission risk assessment system 130 can link the patient data to unique non-personal patient identifiers associated with each patient in the patient data. In some embodiments, the linking process can be performed using one or more of the embodiments of U.S. application Ser. No. 17/018,953 filed on Sep. 11, 2020, and entitled “Single Identifier Platform for Storing Entity Data,” which is hereby incorporated by reference herein in its entirety. At block 458, the readmission risk assessment system 130 can calculate a patient readmission risk score for each patient in the patient data using a patient readmission risk model. At block 458, the readmission risk assessment system 130 can also generate actionable insights associated with SDOH that indicate why each of the patients has received the respective the readmission risk score as well as recommendations for patient engagement strategies based on the respective readmission risk score or the actionable insights to help prevent the negative health outcome for the respective patient. In some embodiments, the scoring process can be performed using the embodiments of FIG. 12A and FIG. 12B. At block 460, the method 450 can end. Embodiments of the processes associated with linking the historical claims data to unique non-personal patient identifiers and calculating a patient readmission risk score using a patient readmission risk model will be further described herein.

### Data Linking and Aggregation

FIG. 5 is a flow diagram illustration an example embodiment of a method 500 for linking and aggregating historical claims data associated with patients 110.

The linking or pinning process can be implemented by associating the historical claims data to a non-personal, unique identifiers of the patients 110. The historical claims data may be provided to the readmission risk assessment system 130 by the client systems 120 via the network 140. Tables 3 and 4 shown below show the historical claims data shown in Tables 1 and 2 with personal information scrubbed (for example, removed) and replaced with unique, non-personal identifier associated with each patient.

TABLE 3
Example Pinned Historical Claims Data for the First Provider
Claims Data - Hospital A

| | | | | Primary | | | |
|---|---|---|---|---|---|---|---|
| | | Client | | Diagnosis | | Discharge | |
| PIN | HH ID | ID | County | Code | Admit Date | Date | . . . |
| X5M | 2948 | H-A | LA | C2039 Sepsis | Sep. 1, 2019 | Sep. 6, 2019 | . . . |
| VX5 | 1283 | H-A | LA | C2044 Liver | Jan. 10, 2018 | Jan. 31, 2018 | . . . |
| | | | | Infection | | | |
| . . . | . . . | H-A | . . . | . . . | . . . | . . . | . . . |

TABLE 4
Example Pinned Historical Claims Data for the Second Provider
Claims Data - Hospital B

| | | | | Primary | | | |
|---|---|---|---|---|---|---|---|
| | | Client | | Diagnosis | | Discharge | |
| PIN | HH ID | ID | County | Code | Admit Date | Date | . . . |
| X5M | 2948 | H-B | LA | D2839 Infection | Sep. 20, 2019 | Sep. 21, 2019 | . . . |
| 7BP | 5549 | H-B | LA | C2044 Liver | Jan. 10, 2018 | Jan. 13, 2018 | |
| | | | | Infection | | | |
| . . . | . . . | H-B | . . . | . . . | . . . | . . . | . . . |

As shown in Tables 3 and 4, historical claims data (for example, primary diagnosis code, admission date, and discharge date) is now associated with a unique non-personal identifier for each patient in the historical claims data that is in turn may be associated with non-regulated SDOH data for respective patients. This allows the readmission risk assessment system 130 to generate links between historical claims data and non-regulated SDOH data for patients and find correlation between, for example, readmission and non-regulated SDOH data (for example, age, potential financial hardships, potential lack of transportation, and so forth).

The method 500 can begin at block 502. At block 504, the readmission risk assessment system 130 can receive or access historical claims data. As discussed herein, the historical claims data includes data associated with the patients 110 who received services (for example, medical services) from the clients associated with client systems 120 (for example, medical care providers such as hospitals). At block 506, the readmission risk assessment system 130 can assign a unique identifier to each of the records. The unique identifier may be non-personal so that it cannot identify a person associated with a given record. At block 508, the readmission risk assessment system 130 can aggregate clusters associated with different client network servers. For example, each of the clusters generated at block 508 may be for each individual client network server, each associated with a given client of the client systems 120. As such, there may be one or more clusters from different client network servers that are associated with the same entity, and those clusters (associated with the same entity but from different client network servers) may be aggregated to, for example, get a more complete understanding of a given entity (for example, a patient).

Table 5 below shows aggregated and pinned data of Tables 3 and 4. The example aggregated data shown in Table 5 includes data from two hospitals regarding multiple patients, at least one of which was admitted at both Hospital A and Hospital B.

TABLE 5
Example Aggregated Pinned Historical Claims Data
Combined Claims Data - Hospital A + Hospital B

| | | | | Primary | | | |
|---|---|---|---|---|---|---|---|
| | | Client | | Diagnosis | | Discharge | |
| PIN | HH ID | ID | County | Code | Admit Date | Date | . . . |
| X5M | 2948 | H-A | LA | C2039 Sepsis | Sep. 1, 2019 | Sep. 6, 2019 | . . . |
| | | H-B | LA | D2839 Infection | Sep. 20, 2019 | Sep. 21, 2019 | . . . |
| | | H-F | LA | C2839 Eye | Dec. 20, 2019 | Dec. 20, 2019 | |
| | | | | Infection | | | |
| VX5 | 1283 | H-A | LA | C2044 Liver | Jan. 10, 2018 | Jan. 31, 2018 | . . . |
| | | | | Infection | | | |
| 7BP | 5549 | H-B | LA | C2839 Eye | Jan. 10, 2018 | Jan. 31, 2018 | |
| | | | | Infection | | | |
| . . . | . . . | H-A | . . . | . . . | . . . | . . . | . . . |

Additional information regarding data linking or pinning can be found in U.S. patent application Ser. No. 17/018,953, filed Sep. 11, 2020, titled SINGLE IDENTIFIER PLATFORM FOR STORING ENTITY DATA, which is incorporated herein by reference in its entirety. For example, unique identifiers can be generated by generating pairs of records from the historical claims data. The readmission risk assessment system 130 can calculate a score for each pair of records. The score for each pair of records can represent a likelihood or a probability that a given pair of records contain data associated with the same entity (for example, a patient). The readmission risk assessment system 130 can determine clusters of one or more records. Each of the clusters of one or more records can be associated with an entity (for example, a patient). The readmission risk assessment system 130 can generate a unique identifier for each of the clusters. The unique identifier may be non-personal so that it cannot identify a person associated with a given cluster.

FIG. 6 is a schematic diagram illustrating an embodiment of the method 500 for linking or pinning the historical patient data to replace the personally identifiable data with a unique identifier and then aggregating patient information from multiple clients. As discussed herein, historical claims data 600 may be collected and stored in different client network servers. The historical claims data 600 may be sent to or accessed by the readmission risk assessment system 130 for developing a readmission risk model. The data aggregator 200 of the readmission risk assessment system 130 can receive or access the historical claims data 600 and generate aggregated data 600 using, for example, a technique outlined by the method 500. As shown in FIG. 6, each record or group in the aggregated data 610 may be associated with a unique non-personal identifier associated with a unique individual. The non-personal identifier associated with each individual may also be used to store and manage non-regulated data, such as SDOH data or marketing data.

### Readmission Tagging

FIG. 7 is a flow diagram illustrating an example embodiment of a method 700 for assigning readmission tags. The method 700 can begin at block 702. At block 704, the readmission risk assessment system 130 can assess historical claims data associated with a unique identifier (for example, the aggregated data 610 shown in FIG. 6). At block 706, the readmission risk assessment system 130 can identify a first admission data of a first admission associated with the unique identifier. At block 708, the readmission risk assessment system 130 can identify a second admission data of a second admission associated with the unique identifier. At block 710, the readmission risk assessment system 130 can compare the first admission data and the second admission data. At block 712, the readmission risk assessment system 130 can assign a readmission tag based on the comparison between the first admission data and the second admission data. The readmission tag can be assigned to one of the first admission and the second admission, or to both. For example, if the second admission occurred after the first admission, only the second admission may be assigned as a readmission.

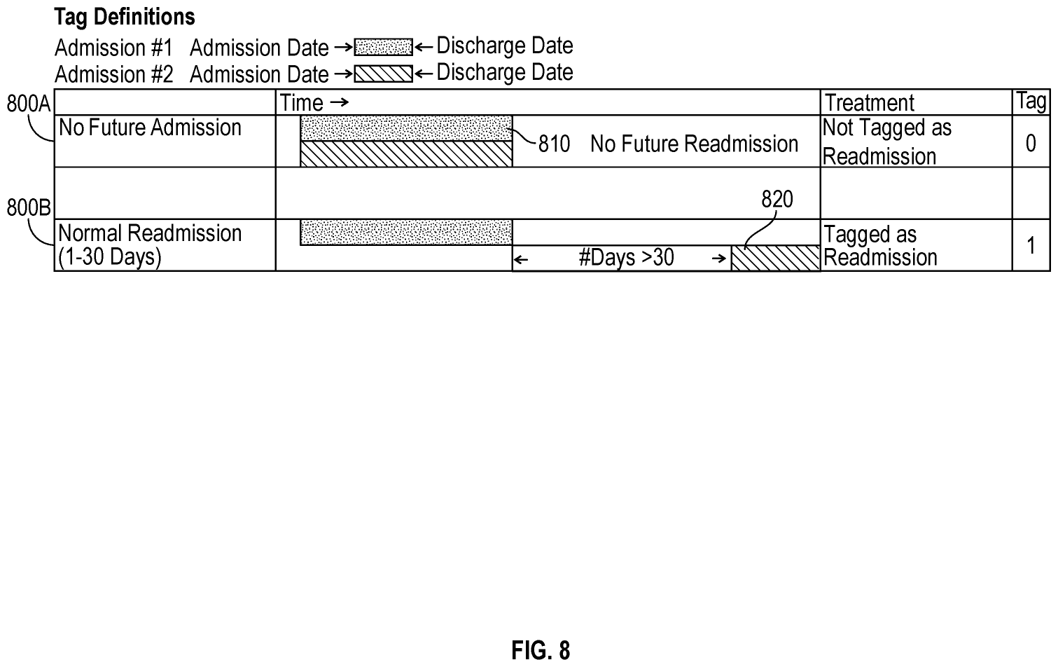

FIG. 8 schematically illustrates embodiments of different scenarios of determining whether to tag an admission as a readmission. In scenario 800A, the first admission 810 is present but there is no second admission. As such, the readmission risk assessment 130 may determine that there is no readmission. In scenario 800B, the first admission 810 and the second admission 820 may be separated by less than a predetermined number of days. In the illustrated embodiment, the predetermined number of days may be 30 days. The predetermined number of days may be greater than or less than 30 days. The predetermined number of days may be adjusted by the client systems 120 or the readmission risk assessment system 130 and different predetermined numbers may be used for different models and/or providers. As such, the second admission may be tagged as a readmission.

It is recognized that FIG. 8 illustrates embodiments of readmission categories, tags, and rules for tagging and/or considering admission data from generating the readmission risk model, and that other embodiments may be used that include different set of readmission tags, rules for tagging, and/or rules for considering admission data.

Table 6 below shows example tags that can be applied to linked or pinned historical claims data that are used in FIG. 8 where different tags are assigned to one or more categories.

TABLE 6
Example Readmission Tags
Tagging Key

| Tag | Category |
|---|---|
| 0 | No future readmission |
| 1 | Readmission between 1-30 days |

Table 7 below shows example readmission categories and tags applied to the aggregated pinned historical claims data of Table 5.

TABLE 7
Tagged Pinned Historical Claims Data
Combined Claims Data - Hospital A + Hospital B

| | | | | Primary | | | | Days from | | |
|---|---|---|---|---|---|---|---|---|---|---|
| | | Client | | Diagnosis | | Discharge | Length | Prev. | Readmission | |
| PIN | HH ID | ID | County | Code | Admit Date | Date | of Stay | Admission | Category | Tag |
| X5M | 2948 | H-A | LA | C2039 Sepsis | Sep. 1, 2019 | Sep. 6, 2019 | 5 | None | Readmission | 1 |
| | | | | | | | | | between 1-30 | |
| | | | | | | | | | days | |
| | | H-B | LA | D2839 Infection | Sep. 20, 2019 | Sep. 21, 2019 | 1 | 14 | No future | 0 |
| | | | | | | | | | readmission | |
| | | H-F | LA | C2839 Eye | Dec. 20, 2019 | Dec. 20, 2019 | 0 | 90 | No future | 0 |
| | | | | Infection | | | | | readmission | |
| VX5 | 1283 | H-A | LA | C2044 Liver | Jan. 10, 2018 | Jan. 31, 2018 | 21 | None | No future | 0 |
| | | | | Infection | | | | | readmission | |
| 7BP | 5549 | H-B | LA | C2839 Eye | Jan. 10, 2018 | Jan. 31, 2018 | 3 | None | No future | 0 |
| | | | | Infection | | | | | readmission | |
| . . . | | H-A | . . . | . . . | . . . | . . . | . . . | . . . | . . . | . . . |

### Generating Risk Tables

FIG. 9 is a flow diagram illustrating an example embodiment of a method 900 for generating one or more risk tables. The method 900 can begin at block 902. At block 904, the readmission risk assessment system 130 can identify one or more categories of tagged historical claims data. In some embodiments, the tagged historical data may be generated by using methods described with respect to FIG. 7. The tagged historical claims data may include admissions data (for example, admission dates, discharge dates, diagnosis, and so forth) that can be tagged as a readmission. At block 906, the readmission risk assessment system 130 can aggregate the tagged data into predictive segments based on the one or more categories. Table 8 below shows example tagged data of Table 7 aggregated into predictive segments. In the example shown in Table 8, the predictive segments are clustered based on mapped diagnosis code associated with each claim data. The mapped diagnosis codes can represent one or more diagnoses. For example, one or more diagnoses can be grouped based on outcome similarities (for example, similar patient admission duration) and be mapped together to a single mapped diagnosis code (for example, unique code). The mapped diagnosis code can be generated based on existing diagnosis codes (for example, S065X0A and A419 as shown in FIG. 10) or randomly generated. By aggregating the tagged data into predictive segments such as the mapped diagnostic codes, the readmission risk assessment system 130 can provide, for example, correlation information between readmission rate (for example, determined from readmission tags) and those predictive segments (for example, mapped diagnosis codes, types of diagnosis, degree of patient risk, estimated cost of care, and so forth).

TABLE 8
Example Tagged Data Aggregated into Predictive Segments
Combined Claims Data - Hospital A + Hospital B

| | | | | Mapped | | | | Days from | | |
|---|---|---|---|---|---|---|---|---|---|---|
| | | Client | | Diagnosis | | Discharge | Length | Prev. | Readmission | |
| PIN | HH ID | ID | County | Code | Admit Date | Date | of Stay | Admission | Category | Tag |
| X5M | 2948 | H-A | LA | X037 | Sep. 1, 2019 | Sep. 6, 2019 | 5 | None | Readmission | 1 |
| | | | | | | | | | between 1-30 | |
| | | | | | | | | | days | |
| | | H-B | LA | X135 | Sep. 20, 2019 | Sep. 21, 2019 | 1 | 14 | No future | 0 |
| | | | | | | | | | readmission | |
| | | H-F | LA | X037 | Dec. 20, 2019 | Dec. 20, 2019 | 0 | 90 | No future | 0 |
| | | | | | | | | | readmission | |
| VX5 | 1283 | H-A | LA | X037 | Jan. 10, 2018 | Jan. 31, 2018 | 21 | None | No future | 0 |
| | | | | | | | | | readmission | |
| 7BP | 5549 | H-B | LA | X037 | Jan. 10, 2018 | Jan. 31, 2018 | 3 | None | No future | 0 |
| | | | | | | | | | readmission | |
| . . . | | H-A | . . . | . . . | . . . | . . . | . . . | . . . | . . . | . . . |

At block 908, the readmission risk assessment system 130 can generate one or more risk tables based on the aggregated tagged data where each of the risk tables are based on one or more predictive criteria. The method 900 can end at block 910.

FIG. 10 is a schematic diagram illustrating an example embodiment of a process for generating one or more risk tables. In the illustrated embodiment, historical claims data can include diagnostic information (for example, “03411 (Malignant neoplasm of upper lobe, right bronchus or lung)” or “A419 (Sepsis)”) and readmission risk information. The readmission risk assessment system 130 can, based on the historical claims data, generate readmission risk level data 1010. In the illustrated embodiment, the readmission risk level data 1010 can indicate various readmission risk levels for different categories of, for example, diagnoses. The categories may be predetermined by the readmission risk assessment system 130 or the client systems 120. Alternatively, the categories may be adjustable and/or based on different criteria. The readmission risk assessment system 130 can take a portion of the readmission risk level data 1010 for generating one or more risk tables. For example, the portion of the readmission risk level data 1010 used for generating the one or more risk tables may represent a predetermined time period (for example, 24 months, 30 months, 48 weeks). The predetermined time period for generating the one or more risk tables may be changed by the readmission risk assessment system 130 and/or the client systems 120. An aggregator (for example, the data aggregator 220) 1030 may collect portions of the readmission risk level data 1010 from the client systems 120 and generate one or more risk tables 1040. The risk tables 1040 may include one or more categories 1042 and indicators 1044 that may represent readmission risk levels. The readmission risk levels may be indicated using at least one of different colors, different shading, or alphanumeric characters.

FIG. 11 is an example embodiment of the risk table 1040 based on mapped diagnosis codes. The risk table 1040 can include the categories 1042. The risk table shown in FIG. 11 may be prepared by determining a total number of claims, a number of readmissions for each segment (for example, “Mapped Diagnosis Code”), and calculating a readmission risk for each segment based on the total number of claims and a number of readmissions for each segment. For each of the categories, a total number of claims 1110 (for example, “495,732”), a number of readmissions 1120 (for example, “90,223”), and a readmission rate 1130 (for example, “18.2%”) can be provided. In some embodiments, a description could also be provided. The readmission rate 1130 may be calculated based on the total number of claims 1110 and the number of readmissions 1120. In the illustrated embodiment, the readmission rate 1130 for a given category is calculated by dividing the number of readmissions 1120 for the given category by the total number of claims 1110 for the given category. In some embodiments, the risk table 1040 may represent historical claims data and readmission data for a single client or more than one clients.

Tables 9 and 10 below show some other example risk tables generated based on data included in Table 8. In Table 9, the tagged pinned historical claim data is categorized (or segmented) using “County” (for example, where a given patient lives). As shown in Table 10, more than one predictable segments may be used to generate one or more risk tables. In the illustrated embodiment, the tagged pinned historical claims data shown in Table 10 are categorized (or segmented) using “Mapped Diagnosis Code” and “Days from Previous Admission”).

TABLE 9
Example Categorized Tagged Historical Claims Data
Combined Claims Data - Hospital A + Hospital B

| | # of | # of | Readmission |
|---|---|---|---|
| County | Claims | Readmissions | Rate |
| . . . | . . . | . . . | . . . |
| LA | 65122 | 22150 | 34.0% |
| Orange | 51565 | 13512 | 26.2% |
| Riverside | 15681 | 6458 | 22.1% |
| San Diego | 12652 | 4415 | 34.9% |
| . . . | . . . | . . . | . . . |

TABLE 10
Example Categorized Tagged Historical Claims Data
Combined Claims Data - Hospital A + Hospital B

| Mapped | Days from | | | |
|---|---|---|---|---|
| Diagnosis | Previous | # of | # of | Readmission |
| Code | Admission | Claims | Readmissions | Rate |
| X123 | All | 495,732 | 90,223 | 18.2% |
| | None | 118,729 | 12,761 | 10.7% |
| | 0 | 129,837 | 24,267 | 18.7% |
| | 30 | 158,375 | 39,365 | 24.9% |
| | 60 | 41,936 | 4,135 | 9.9% |
| | 90 | 28,735 | 3,753 | 13.1% |
| | Other | 18,120 | 5,942 | 32.8% |
| X135 | All | 54,939 | 4,340 | 7.9% |
| | None | 14,830 | 735 | 5.0% |
| | 0 | 11,837 | 1,374 | 11.6% |
| | 30 | 8,293 | 816 | 9.8% |
| | 60 | 9,527 | 573 | 6.0% |
| | 90 | 8,441 | 642 | 7.6% |
| | Other | 2,011 | 170 | 8.5% |
| . . . | . . . | . . . | . . . | . . . |

While example categories have been used, it is recognized that a wide variety of predictive categories may be used to generate the risk tables.

### Calculating Readmission Risk Scores

FIG. 12A is a flow diagram illustrating an example embodiment of a method 1200 for calculating a patient readmission risk score using a deployed readmission risk score model. The method 1200 can begin at block 1202. At block 1204, the readmission risk assessment system 130 accesses patient record (for example, patient admission data) associated with a patient that is about to be or has been recently discharged. For example, the patient record may include admission data, discharge data, diagnosis data, name, age, gender, county of residence and any other types of information that may be collected by a care provider from a patient. An example of the patient record received by the readmission risk assessment system 130 is shown below in Table 11.

TABLE 11
Example Patient Record

| Client | First | Last | | | |
|---|---|---|---|---|---|
| ID | Name | Name | Date of Birth | County | . . . |
| H-A | John | Smith | May 11, 1980 | LA | . . . |

At block 1206, the readmission risk assessment system 130 can determine a unique identifier for the patient. The unique identifier may be one previously associated with the patient for non-regulated data purposes. An example of the patient record with a unique identifier for a patient is shown below in Table 12.

TABLE 12
Example Patient Record with Unique Identifier

| Client | | | First | Last | Date of | |
|---|---|---|---|---|---|---|
| ID | PIN | HH ID | Name | Name | Birth | County |
| H-A | X5M | 2948 | John | Smith | May 11, 1980 | LA |

At block 1208, the readmission risk assessment system 130 can access other data sources (for example, the third-party database 152 of the third-party system 150) and retrieve (or receive) non-regulated SDOH data (for example, demographic data) associated with the patient. In some embodiments, the non-regulated SDOH data may be retrieved or received using the unique identifier associated with the patient. The non-regulated SDOH data may be provided at an individual-level (for example, whether a patient has a college degree) or at a household-level (for example, how many people in the patient's household has a college degree). Some examples of non-regulated SDOH data associated with a unique non-personal identifier are shown below in Tables 13 and 14.

TABLE 13
Example SDOH Data (Individual-Level)

| PIN | HH ID | College Degree? | Rent v. Own |
|---|---|---|---|
| X5M | 2948 | Yes | Rent |

TABLE 14
Example SDOH Data (Household-Level)

| | # People In HH |
|---|---|
| HH ID | With College Degree |
| 2948 | 2 |

The retrieved non-regulated SDOH data then can be appended to the patient records as shown in Table 15 below.

TABLE 15
Example Patient Record with SDOH Data

| | | | | | | # people in |
|---|---|---|---|---|---|---|
| Client | | | | College | Rent v. | household with |
| ID | PIN | HH ID | County | Degree? | Own | College Degree |
| H-A | X5M | 2948 | LA | Yes | Rent | 2 |

In some embodiments, credit, and financial information such as loan-to-value ratio and total spending in last X number of days (for example, 10 days) may be included in the non-regulated SDOH data and appended to the patient records. An example patient record appended with such information is shown in Table 16 below.

TABLE 16
Another Example Patient Record with SDOH Data

| | | | | | | | | Current |
|---|---|---|---|---|---|---|---|---|
| | | | | | | # People In | Total Dollars | Loan To |
| Client | | | | College | Rent v. | Household With | Spent In Last | Value |
| ID | PIN | HH ID | County | Degree? | Own | College Degree | 10 Days | Ratio |
| H-A | X5M | 2948 | LA | Yes | Rent | 2 | $520.25 | 65% |

In some embodiments, after retrieving or receiving the non-regulated SDOH data, the readmission risk assessment system 130 can, at block 1210, feed the patient record and the demographic data into a readmission risk model. Alternatively, the readmission risk assessment system 130 can in real-time, at block 1212, query a client system for patient information (for example, duration of admission, mapped diagnosis code, symptoms, and so forth). An example of the queried data is shown below in Table 17.

TABLE 17
Example Queried Data Received from a Care Provider

| Mapped Diagnosis Code | # of Days |
|---|---|
| X123 | 3 |

After the readmission risk assessment system 130 receives the queried data, the readmission risk assessment system 130 can, at block 1214, run a readmission risk model using the patient record, the demographic data, and/or the queried data.

After running the readmission risk model (with or without the queried data from the client system), the readmission risk assessment system 130 can, at block 1216, return an updated (for example, including a readmission risk score) patient record with a readmission risk score. An example of the updated patient record with a readmission risk score is shown in Table 18 below. The method 1200 can end at block 1218.

TABLE 18
Example Updated Patient Record with Readmission Risk Score

| | | | | | # People In | | # People In |
|---|---|---|---|---|---|---|---|
| Client | | | | College | Household With | Rent V. | Household With |
| ID | PIN | HH ID | County | Degree? | College Degree | Own | College Degree |
| H-A | X5M | 2948 | LA | Yes | 2 | Rent | 2 |

| Total $ Spent | Current Loan | Readmission | SDOH Factor: | SDOH Factor: |
|---|---|---|---|---|
| In Last 10 Days | To Value Ratio | Risk Score | Access To Care | Access Medication |
| $520.25 | 65% | 778 | 388 | 226 |

FIG. 12B is a flow diagram illustrating another example embodiment of a method 1250 for calculating a patient readmission risk score. The method 1250 can begin at block 1252. At block 1254, the readmission risk assessment system 130 can determine whether the readmission risk assessment system 130 received patient claims data (for example, patient admission or claims data) from a client network server. If the readmission risk assessment system 130 determines that it did not receive patient claims data from the client network server, then the method 1250 can proceed to block 1256, at which the readmission risk assessment system 130 can determine whether it received non-regulated SDOH data associated with the patient from the third-party server. If the readmission risk assessment system 130 determines that it did not receive the non-regulated SDOH data, the method 1250 can end at block 1260. If the readmission risk assessment system 130 determines that it received non-regulated SDOH data, the method can proceed to block 1258, at which the readmission risk assessment system 130 can calculate a readmission risk score for a patient based on just the non-regulated SDOH data. After determining the readmission risk score based at block 1258, the method 1250 can end at block 1260.

If the readmission risk assessment system 130, at block 1254, determines that it received patient claims data from the client network server, the method can proceed to block 1262. At block 1262, the readmission risk assessment system 130 can determine whether it received non-regulated SDOH data from the third-party server. If the readmission risk assessment system 130 determines that it received non-regulated SDOH data from the third-party server, then the readmission risk assessment system 130 can, at block 1264, calculate a readmission risk score for the patient based on the patient claims data and the non-regulated SDOH data. However, if the readmission risk assessment system 130 determines that it did not receive non-regulated SDOH data from the third-party server, then the readmission risk assessment system 130 can, at block 1268, calculate a readmission risk score for the patient based on the patient claims data. The method 1250 can end at block 1266.

FIG. 13 is a flow diagram illustrating an example embodiment of a method 1300 for running a readmission risk model in different scenarios. At block 1302, a care provider (for example, a nurse or a doctor) accesses patient information at discharge. At block 1304, a user interface is provided to a device being utilized by the care provider. The user interface can allow the care provider to provide various information about a patient being discharged. For example, in the illustrated embodiment, the user interface may prompt the care provider to provide information about the patient being discharged that is associated with the predictive categories including, for example, diagnosis code (for example, A419) and/or admission duration (for example, 3 days). At block 1306A, the care provider may follow a normal discharge process and, via the user interface, submit a diagnosis code for the patient being discharged. At block 1306B, the care provider may submit, via the user interface, an admission duration for the patient being discharged. It is contemplated that the care provider may provide one or the other, or both. User interfaces 1308A, 13088 can include a prompt for the care provider (“How many days did the patient stay in hospital?”) and a display of available answer options or an entry component for entering in the requested information. The readmission risk assessment system 130 then uses the provided information to determine the corresponding risk level. For example, if the care provider inputs diagnosis code A419 and a 3 day patient stay, the readmission risk assessment system 130 may feed the diagnosis code into a translator that uses the diagnosis code to determine the appropriate mapped diagnosis code (for example, X123), and then the readmission risk assessment system 130 uses the mapped diagnosis code along with the other provided information to determine the readmission rate. If the care provider is able to provide information at discharge (for example, information related to diagnosis or admission duration), the readmission risk assessment system 130 can, at block 1310, run a readmission risk model using risk attributes based on historical claim data and SDOH attributes based on non-regulated SDOH data. Alternatively, if the care provider does not provide information at discharge, the readmission risk assessment system 130 can, at block 1312, run a readmission risk model using information available to the care provider, such as, non-regulated SDOH data (for example, age, income level, home address, and so forth). In some embodiments, the readmission risk assessment system 130 uses information already provided during discharge, but in other embodiments, additional prompts can be added to the user interface to request additional information from the care provider.

### Example User Interface

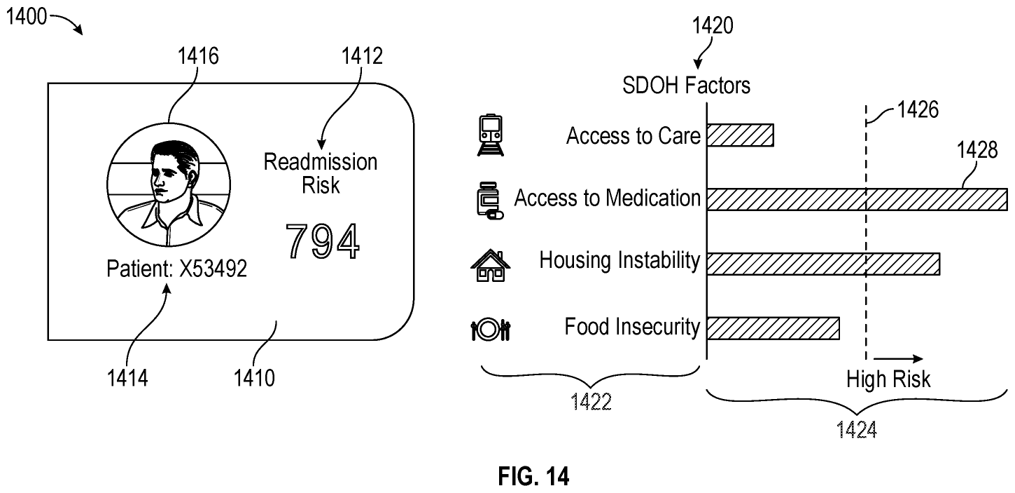

FIG. 14 illustrates an embodiment of an example user interface 1400 for displaying a patient readmission risk score and other information related to the patient readmission risk score. The user interface 1400 can include a patient section 1410 and a risk factors section 1420.

The patient section 1410 can include a readmission risk score 1412, a unique identifier 1414, and a patient visual 1416 associated with a patient. The unique identifier 1414 may be the unique identifier used to retrieve or receive non-regulated SDOH data associated with the patient. Additionally, the unique identifier 1414 may be the unique identifier used to group historical claims data between one or more clients. The patient visual 1416 may be, as shown in FIG. 14, a picture of the patient. Additionally or alternatively, the patient visual 1416 may include a name of the patient.

The risk factors section 1420 can include one or more SDOH factors 1422 and risk scores section 1424. The SDOH factors 1422 may be factors or actionable insights that impact a readmission risk score for a given patient. In the illustrated embodiment, the SDOH factors 1422 include “Access to Care,” “Access to Medication,” “Housing Instability,” and “Food Insecurity.” For each SDOH factor 1422, a graphical display element 1428 (for example, a bar graph) may be generated in the risk scores section 1424. The graphical display element 1428 may represent a risk score for each SDOH factor 1422. The risk score (or the graphical display element 1428) for each SDOH factor 1422 may be compared to a predetermined threshold 1426 that may be representative of a threshold value for distinguishing between low and high risks. For example, as shown in FIG. 14, the graphical displays indicate that risk levels for “Access to Care” and “Food Insecurity” is below the threshold 1426, while risk levels for “Access to Medication” and “Housing Instability” is above the threshold 1426. This may indicate that “Access to Medication” and “Housing Instability” may be larger factors that lead to, for example, a high readmission risk score of a patient displayed in the patient section 1410.

In some embodiments, the readmission risk assessment system 130 may also generate and provide recommendations for patient engagement strategies based on the readmission risk score 1412 or the SDOH factors 1422 to help prevent the negative health outcome. The readmission risk assessment system 130 may include or access a datastore that includes a set of recommendations as well as a rule set for selecting and prioritizing the recommendations. In some embodiments, the readmission risk assessment system 130 may include instructions for automatically generating instructions for enrolling the patient into a specific program based on the readmission risk score 1412 or the SDOH factors 1422. For example, if the access to medication SDOH factor 1422 indicates a high risk, readmission risk assessment system 130 may generate an enrollment data package to be automatically submitted to system that handles mail order medication distribution. In addition, the readmission risk assessment system 130 may be configured to provide automated alerts if the readmission risk score 1412 or the SDOH factors 1422 are above predetermined thresholds and the user interface may be instructed to highlight the readmission risk score 1412 or the SDOH factors 1422 that are above the predetermined thresholds, such as via a user interface element (for example, red bold font, flashing text, an audible beep, and so forth).

### Computer Systems

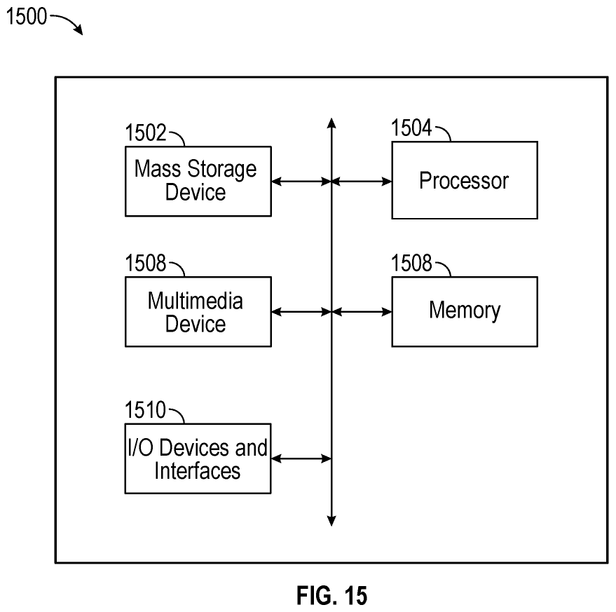

FIG. 15 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 15. While FIG. 15 illustrates an embodiment of a computing system 1500, it is recognized that the functionality provided for in the components and modules of computer system 1500 may be combined into fewer components and modules, or further separated into additional components and modules.

The computer system 1500 can comprise a processor 1504 that carries out the functions, methods, acts, modules, and/or processes described herein. The processor 1504 can be a central processing unit (CPU) or a graphics processing unit (GPU).

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 1500 can include a physical memory 1508, such as random-access memory (RAM) for temporary storage of information, a read only memory (ROM) for permanent storage of information, and a mass storage device 1502, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. The components of the computer system 1500 may be connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 1500 can include one or more input/output (I/O) devices and interfaces 1510, such as a keyboard, mouse, touch pad, and printer. The I/O devices and interfaces 1510 can include one or more display devices, such as a monitor, that allows the visual presentation of data to a user. More particularly, a display device provides for the presentation of GUIs as application software data, and multi-media presentations, for example. The I/O devices and interfaces 1510 can also provide a communications interface to various external devices. The computer system 1500 may comprise one or more multi-media devices 1508, such as speakers, video cards, graphics accelerators, and microphones, for example.

The computer system 1500 may run on a variety of computing devices, such as a server, a Windows server, a Structure Query Language server, a Unix Server, a personal computer, a laptop computer, and so forth. In other embodiments, the computer system 1500 may run on a cluster computer system, a mainframe computer system and/or other computing system suitable for controlling and/or communicating with large databases, performing high volume transaction processing, and generating reports from large databases. The computing system 1500 is generally controlled and coordinated by an operating system software, such as Windows XP, Windows Vista, Windows 7, Windows 8, Windows 10, Windows 11, Windows Server, Unix, Linux (and its variants such as Debian, Linux Mint, Fedora, and Red Hat), SunOS, Solaris, Blackberry OS, z/OS, iOS, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 1500 illustrated in FIG. 15 can be coupled to a network, such as a LAN, WAN, or the Internet via a communication link (wired, wireless, or a combination thereof). The network may communicate with various computing devices and/or other electronic devices. Additionally, the network can communicate with one or more other computing systems (for example, external computing systems) and one or more data sources (for example, external data sources). Connections may be a direct physical connection, a virtual connection, and other connection type. The web-enabled user access point may comprise a browser module that uses text, graphics, audio, video, and other media to present data and to allow interaction with data via the network.

Access to the computer system 1500 by other computing systems and/or by data sources may be through a web-enabled user access point such as the computing systems' or data source's personal computer, cellular phone, smartphone, laptop, tablet computer, e-reader device, audio player, or another device capable of connecting to the network. Such a device may have a browser module that is implemented as a module that uses text, graphics, audio, video, and other media to present data and to allow interaction with data via the network.

The output module may be implemented as a combination of an all-points addressable display such as a cathode ray tube (CRT), a liquid crystal display (LCD), a plasma display, or other types and/or combinations of displays. The output module may be implemented to communicate with input devices 1510 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the computer system 1500 may comprise a physical or logical connection established between a remote microprocessor and a mainframe host computer for the express purpose of uploading, downloading, or viewing interactive data and databases on-line in real time. The remote microprocessor may be operated by an entity operating the computer system 1500, including the client server systems or the main server system, an/or may be operated by one or more of the data sources and/or one or more of the other computing systems. In some embodiments, terminal emulation software may be used on the microprocessor for participating in the micro-mainframe link.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 1500 may include one or more internal and/or external data sources. In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Cache), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MongoDB Atlas, Amazon Aurora, Amazon DynamoDB, Amazon Redshift, Amazon ElastiCache, Amazon MemoryDB for Redis, Amazon DocumentDB, Amazon Keyspaces, Amazon Neptune, Amazon Timestream, or Amazon QLDB), a non-relational database, or a record-based database.

The computer system 1500 may also access one or more databases. The databases may be stored in a database or data repository. The computer system 1500 may access the one or more databases through a network or may directly access the database or data repository through I/O devices and interfaces 1510. The data repository storing the one or more databases may reside within the computer system 1500.

### Additional Embodiments

Each of the processes, methods, and algorithms described in the preceding sections may be embodied in, and fully or partially automated by, code modules executed by one or more computer systems or computer processors comprising computer hardware. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

The various features and processes described above may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

As used herein, the terms “determine” or “determining” encompass a wide variety of actions. For example, “determining” may include calculating, computing, processing, deriving, generating, obtaining, looking up (for example, looking up in a table, a database, or another data structure), ascertaining and the like via a hardware element without user intervention. Also, “determining” may include receiving (for example, receiving information), accessing (for example, accessing data in a memory) and the like via a hardware element without user intervention. Also, “determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (for example, transmitting or receiving) information. A message may include a machine readable aggregation of information such as an XML document, fixed field message, comma separated message, or the like. A message may, in some implementations, include a signal utilized to transmit one or more representations of the information. While recited in the singular, it will be understood that a message may be composed, transmitted, stored, received, and so forth, in multiple parts.

As used herein “receive” or “receiving” may include specific algorithms for obtaining information. For example, receiving may include transmitting a request message for the information. The request message may be transmitted via a network as described above. The request message may be transmitted according to one or more well-defined, machine readable standards which are known in the art. The request message may be stateful in which case the requesting device and the device to which the request was transmitted maintain a state between requests. The request message may be a stateless request in which case the state information for the request is contained within the messages exchanged between the requesting device and the device serving the request. One example of such state information includes a unique token that can be generated by either the requesting or serving device and included in messages exchanged. For example, the response message may include the state information to indicate what request message caused the serving device to transmit the response message.

As used herein “generate” or “generating” may include specific algorithms for creating information based on or using other input information. Generating may include retrieving the input information such as from memory or as provided input parameters to the hardware performing the generating. After obtained, the generating may include combining the input information. The combination may be performed through specific circuitry configured to provide an output indicating the result of the generating. The combination may be dynamically performed such as through dynamic selection of execution paths based on, for example, the input information, device operational characteristics (for example, hardware resources available, power level, power source, memory levels, network connectivity, bandwidth, and the like). Generating may also include storing the generated information in a memory location. The memory location may be identified as part of the request message that initiates the generating. In some implementations, the generating may return location information identifying where the generated information can be accessed. The location information may include a memory location, network locate, file system location, or the like.

As used herein, “activate” or “activating” may refer to causing or triggering a mechanical, electronic, or electro-mechanical state change to a device. Activation of a device may cause the device, or a feature associated therewith, to change from a first state to a second state. In some implementations, activation may include changing a characteristic from a first state to a second state such as, for example, changing the viewing state of a lens of stereoscopic viewing glasses. Activating may include generating a control message indicating the desired state change and providing the control message to the device to cause the device to change state.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

All of the methods and processes described above may be embodied in, and partially or fully automated via, software code modules executed by one or more general purpose computers. For example, the methods described herein may be performed by the computing system and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

What is claimed is:

1. A system for deployment of one or more risk models for assessing patient health outcome risk, the system comprising:
one or more processors;
a network communications interface;
a memory; and
computer code stored in the memory, wherein the computer code, when retrieved from the memory and executed by the one or more processors causes the one or more processors to:
access a first set of electronic patient data associated with a first patient, a provider admission, and an impending discharge event;
automatically generate a linked patient data set based on the first set of electronic patient data and a unique identifier associated with the first patient where the unique identifier does not include any personally identifiable information of the first patient;
associate the linked patient data set with one or more aggregated code indicators generated based on a set of anonymized clinical data comprising an aggregation of a plurality of anonymized provider data sets associated with a plurality of providers, wherein the set of anonymized clinical data includes risk rates for categorized clinical data;
associate the linked patient data set with at least a length of stay based on an admission date associated with the provider admission;
associate the linked data set with marketing attribute data comprising social determinants of health data; and
apply a deployed risk model to generate a risk score associated with a health outcome risk based on the associated one or more aggregated code indicators or the associated marketing attribute data, where in the deployed risk model is generated based on the set of anonymized clinical data, where in the set of anonymized clinical data includes historical clinical data comprising historical data associated with the plurality of providers, historical provider admission data indicating dates of patient admissions associated with the plurality of providers, and historical discharge data indicating dates of patient discharge dates associated with the plurality of providers.

2. The system of claim 1, further comprising computer code stored in the memory, wherein the computer code, when retrieved from the memory and executed by the processor causes the processor to store computer-executable instructions that:
generate instructions for rendering a first user interface component for requesting the one or more aggregated code indicators from a health care provider.

3. The system of claim 1, further comprising computer code stored in the memory, wherein the computer code, when retrieved from the memory and executed by the processor causes the processor to store computer-executable instructions that:
generate instructions for rendering a second user interface component for requesting the length of stay.

4. The system of claim 1, wherein the categorized clinical data is based on one or more of: geographic region, age, diagnosis codes, doctor codes, medication codes, or number of procedures.

5. The system of claim 1, wherein the one or more aggregated code indicators are anonymized clinical data attributes.

6. The system of claim 1, wherein the health outcome risk is readmission risk and further comprising computer code stored in the memory, wherein the computer code, when retrieved from the memory and executed by the processor causes the processor to store computer-executable instructions that:
automatically tag the set of anonymized clinical data to indicate one or more of: no future readmission, or readmission within a predetermined time window.

7. The system of claim 1, further comprising computer code stored in the memory, wherein the computer code, when retrieved from the memory and executed by the processor causes the processor to store computer-executable instructions that:
generate a set of aggregated risk tables configured to store the risk rates for the categorized clinical data.

8. The system of claim 1, wherein the deployed risk model further generates, based on the risk score, one or more of: flagged social determinants of health factors, social determinants of health alerts, actionable social determinants of health insights, or social determinants of health recommendations.

9. The system of claim 1, wherein the health outcome risk is one or more of: a readmission risk, an excessive acute facility utilization risk, an excessive post-acute facility utilization risk, an emergency room frequenter risk, or a high cost of care risk.

10. A computer-implemented method of deploying one or more risk models for assessing patient health outcome risk, the computer-implemented method comprising, as implemented by one or more computing devices configured with specific executable instructions to at least:
access a first set of electronic patient data associated with a first patient, a provider admission, and an impending discharge event;
automatically generate a linked patient data set based on the first set of electronic patient data and a unique identifier associated with the first patient where the unique identifier does not include any personally identifiable information of the first patient;
associate the linked patient data set with one or more aggregated code indicators generated based on a set of anonymized clinical data comprising an aggregation of a plurality of anonymized provider data sets associated with a plurality of providers, wherein the set of anonymized clinical data includes risk rates for categorized clinical data;
associate the linked patient data set with at least a length of stay based on an admission date associated with the provider admission;
associate the linked data set with marketing attribute data comprising social determinants of health data; and
apply a deployed risk model to generate a risk score indicating a health outcome risk based on the associated one or more aggregated code indicators or the associated marketing attribute data, where in the deployed risk model is generated based on the set of anonymized clinical data, where in the set of anonymized clinical data includes historical clinical data comprising historical data associated with the plurality of providers, historical provider admission data indicating dates of patient admissions associated with the plurality of providers, and historical discharge data indicating dates of patient discharge dates associated with the plurality of providers.

11. The computer-implemented method of claim 10 further comprising specific executable instructions that:
generate instructions for rendering a first user interface component for requesting the one or more aggregated code indicators from a health care provider.

12. The computer-implemented method of claim 10 further comprising specific executable instructions that:
generate instructions for rendering a second user interface component for requesting the length of stay.

13. The computer-implemented method of claim 10, wherein the categorized clinical data is based on one or more of: geographic region, age, diagnosis codes, doctor codes, medication codes, or number of procedures.

14. The computer-implemented method of claim 10, wherein the one or more aggregated code indicators are anonymized clinical data attributes.

15. The computer-implemented method of claim 10, wherein the health outcome risk is readmission risk and further comprising specific executable instructions that:
automatically tag the set of anonymized clinical data to indicate one or more of: no future readmission or readmission within a predetermined time window.

16. The computer-implemented method of claim 10 further comprising specific executable instructions that:
generate a set of aggregated risk tables configured to store the risk rates for the categorized clinical data.

17. The computer-implemented method of claim 10, wherein the deployed risk model further generates, based on the risk score, one or more of: flagged social determinants of health factors, social determinants of health alerts, actionable social determinants of health insights, or social determinants of health recommendations.

18. The computer-implemented method of claim 10, wherein the health outcome risk is one or more of: a readmission risk, an excessive acute facility utilization risk, an excessive post-acute facility utilization risk, an emergency room frequenter risk, or a high cost of care risk.

19. A non-transitory computer storage medium storing computer-executable instructions that, when executed by a processor, cause the processor to at least:
access a first set of electronic patient data associated with a first patient, a provider admission, and an impending discharge event;
automatically generate a linked patient data set based on the first set of electronic patient data and a unique identifier associated with the first patient where the unique identifier does not include any personally identifiable information of the first patient;
associate the linked patient data set with one or more aggregated code indicators generated based on a set of anonymized clinical data comprising an aggregation of a plurality of anonymized provider data sets associated with a plurality of providers, wherein the set of anonymized clinical data includes risk rates for categorized clinical data;
associate the linked patient data set with at least a length of stay based on an admission date associated with the provider admission;
associate the linked data set with marketing attribute data comprising social determinants of health data; and
apply a deployed risk model to generate a risk score associated with a health outcome risk based on the associated one or more aggregated code indicators or the associated marketing attribute data, where in the deployed risk model is generated based on the set of anonymized clinical data, where in the set of anonymized clinical data includes historical clinical data comprising historical data associated with the plurality of providers, historical provider admission data indicating dates of patient admissions associated with the plurality of providers, and historical discharge data indicating dates of patient discharge dates associated with the plurality of providers.

20. The non-transitory computer storage medium of claim 19, further storing computer-executable instructions that:
generate instructions for rendering a first user interface component for requesting the one or more aggregated code indicators from a health care provider.

21. The non-transitory computer storage medium of claim 19, further storing computer-executable instructions that:
generate instructions for rendering a second user interface component for requesting the length of stay.

22. The non-transitory computer storage medium of claim 19, wherein the categorized clinical data is based on one or more of: geographic region, age, diagnosis codes, doctor codes, medication codes, or number of procedures.

23. The non-transitory computer storage medium of claim 19, wherein the one or more aggregated code indicators are anonymized clinical data attributes.

24. The non-transitory computer storage medium of claim 19, wherein the health outcome risk is readmission risk and further storing computer-executable instructions that:
automatically tag the set of anonymized clinical data to indicate one or more of: no future readmission or readmission within the predetermined time window.

25. The non-transitory computer storage medium of claim 19, further storing computer-executable instructions that:
generate a set of aggregated risk tables configured to store the risk rates for the categorized clinical data.

26. The non-transitory computer storage medium of claim 19, wherein the deployed risk model further generates, based on the risk score, one or more of: flagged social determinants of health factors, social determinants of health alerts, actionable social determinants of health insights, or social determinants of health recommendations.

27. The non-transitory computer storage medium of claim 19, wherein the health outcome risk is one or more of: a readmission risk, an excessive acute facility utilization risk, an excessive post-acute facility utilization risk, an emergency room frequenter risk, or a high cost of care risk.
